(19) United States
(12) Patent Application Publication
(10) Pub. No.: US 2008/0281281 A1
(43) Pub. Date: Nov. 13, 2008

(54) Vacuum Therapy Device

(76) Inventors: Johannes Meyer, Hamburg (DE); Dirk Baumann, Hamburg (DE)

Correspondence Address: NORTH WEBER & BAUGH LLP, 2479 E. BAYSHORE ROAD, SUITE 707, PALO ALTO, CA, 94303, US

(21) Appl. No.: 11/909,596

(22) PCT Filed: Mar. 22, 2006
(86) PCT No.: PCT/EP06/02623

§ 371 Date: Feb. 5, 2008

(30) Foreign Application Priority Data
Mar. 24, 2005 (DE) 10 2005 014 420.9

(51) Int. Cl. A61M 1/00

(52) U.S. Cl. 604/313; 604/319

(57) ABSTRACT

The invention relates to a therapeutic device to promote the healing of a wound, comprising a porous paid which is permeable for fluids to place on the wound, a bandage to cover the wound and to provide an essentially airtight seal around the wound, a drainage line connecting the pad with a suction pump so that suction can be applied to the wound to draw the fluids away from it, whereby the line is connected to the suction pump via a canister to collect fluid sucked from the wound, a connector to connect the pad to the drainage line, means to determine a prevailing wound pressure basically comprising the wound and this surrounding skin area, as well as a control unit to control the wound pressure. In order to generally improve metrology with the goal of making possible a more reliable and more secure dressing of the wound, and also to design metrology in an especially simple and error-free way, it is proposed that the pad interconnect the suction pump solely via the drainage line.

### CROSS REFERENCE TO RELATED PATENT APPLICATIONS

This application is the National Phase Application under 35 USC §371 of International Application No. PCT/EP2006/002623, filed Mar. 22, 2006, which claims priority to German Patent Application 10 2005 014 420.9, filed Mar. 24, 2005.

### BACKGROUND

A. Technical Field

The present invention relates to a therapeutic device to promote the healing of a wound, comprising:

an essentially airtight cover around the wound, whereby a liquid-permeable pad is preferably provided between the wound and cover, a drainage line connecting the cover with a suction pump,

a drainage line connecting the cover with a suction pump so that the wound can be suctioned to draw fluids from it, whereby a distal end of the drainage line is connected indirectly to the suction pump through a reservoir to collect the fluid suctioned from the wound,

pressure disposal means for the direct or indirect definition of a wound pressure prevailing in the area between the wound and the cover and

a control unit to regulate the wound pressure.

The present invention also relates to a procedure for operating a class-appropriate therapy device.

B. Background of the Invention

Wound healing devices are used to treat secondary healing chronic or acute wounds, in particular in humans. Using the negative pressure present in the skin area around the wound is supposed to remove fluid, that is, in particular wound exudation, from the wound to thereby shorten the healing period. In many cases, the use of such a wound healing device makes wound healing possible at all in the first place.

For example, a class-appropriate wound healing device is described in EP 0 865 304. In the device described therein it is in particular provided that the device comprises an additional line which connects the porous pad to a pressure gage. Using the additional line, it is possible to monitor the pressure at the wound site. For this purpose in the referenced therapy equipment, the pressure sensor will be provided in the housing of the wound healing device. The wound pressure measured by the additional line should be adjusted to the desired value using a control means also provided in the housing.

A disadvantage of this known wound healing device is that it is relatively complex to provide an additional tube line with the drainage line. Such a tube generally increases the complexity of the apparatus. It is true that it has been proposed to use a multi-lumen tube whose main tube is used as a drainage tube and from which a secondary tube is used as a probe tube to measure pressure. However, this results for example in increasing the external diameter of the tube connection between the housing and the wound dressing in an undesirable fashion. It also cannot be excluded that the additional line becomes blocked or e.g. is cut off from the prevailing pressure due to crimping or some other way at the wound site. In such a case, the pressure reading taken by the known therapy device would be incorrect without the cause being an error in the drainage tube or the wound site. Accordingly, the control of the wound pressure would fail.

### SUMMARY OF THE INVENTION

The task of the present invention is therefore to provide a class-appropriate therapy device to promote the healing of a wound in which the metrology is generally improved to make possible a more reliable and secure wound dressing. The invention is also intended to make the metrology of a class-appropriate therapeutic device particularly simple and error-resistant.

This task is solved according to the invention in that the pressure sensor means comprise at least one pressure sensor on the distal end of the drainage tube. By measuring this distal pressure, advantageously an additional line between canister and the wound can be eliminated if the distal pressure can be used in a suitable way as measurement for the wound pressure. This can be done by prior calibration. In this fashion, the control of the wound pressure can be advantageously done in a particularly simple way. That is, according to the invention, beside the drainage line no additional tube line is needed between the pad and the suction pump and/or collecting canister. The result is simpler equipment design. Therefore to determine wound pressure a measuring procedure is used that requires no additional probe tube to the wound area.

The task according to the invention is solved inventively by having the pressure sensor means comprise at least one wound pressure sensor provided under the cover and means for transferring the readings to the control unit. The advantage is that the wound pressure can be measured directly at the wound site. Errors which can occur in the indirect taking of wound pressure are thus advantageously avoided. The application of a pressure sensor directly on the wound site is also advantageous compared to the state of the art procedure for measuring wound pressure in which an additional probe tube is run from the wound to a sensor located in the base unit because pressure gradients can be generated inside the sensor tube which can systematically distort the wound pressure measurement. Instead, according to the present invention, the wound pressure is measured directly at the wound site and transferred to the control unit by, for example, an electric cable or by radio. Overall, according to this feature of the present invention, the error susceptibility of controlling wound pressure can be significantly improved. It is possible, for example, to recognize blockages in the drainage tube if the directly measured wound pressure is compared to another measured quantity, whereby the second measure quantity can be, for example, pump output or a pressure on the distal end of the drainage tube. Similarly, it is possible in this way to recognize a drop in wound pressure due to a crimping of the drainage tube.

According to an advantageous embodiment of the inventive therapeutic device, the means to determine wound pressure comprise at least one strain gage provided in direct proximity to the skin area. Strain gages are robust and therefore particularly suited to being used directly on the patient. In addition to measuring wound pressure, strain gages can also measure patient movement artifacts. Since they produce a signal with a significantly higher frequency than the wound pressure fluctuations, they are easily distinguished from wound pressure variations by the control unit if suitable band pass filters are used. By combining the inventive wound pressure measurement by strain gages at the wound site with other pressure measurement procedures, the only qualitative alternative to reducing strain gage costs is to use them purely qualitatively. In this way, for example, the exceeding of a minimum pressure value can be established only qualitatively. In this way, the cost of the strain gage can be reduced sharply compared to a quantitative evaluated sensor. This is particularly advantageous if all of the components which are in contact with the patient are designed as disposable items so that the strain gage is also a disposable item.

An especially advantageous further development of the invention is obtained by providing the strain gage in an interactive connection with a connector to connect the cover with the drainage line. Such connectors are used in class-appropriate therapy devices in order to obtain a better distribution of the negative pressure input through the drainage tube over the wound. Since the connector is more or less stretched between the drainage tube and the porous paid depending on the negative pressure prevailing on the wound, with the help of the strain gage this measurement of this strain can be used as the measurement for the wound pressure.

For the fine adjustment to various wound pressure levels it is advantageous according to another embodiment of the invention if a control pump is provided in addition to the suction pump. With this configuration, the suction pump which, for example has a substantially greater suction power than the control pump can be used to set a basic pressure value. The higher suction power of the suction pump compared to the suction power of the control pump can be used to extract the wound exudation. As suction pumps with such strong suction power often have the disadvantage of not being able to be finely adjusted or being too sluggish, the use of an additional control pump effectively provides a possibility for fine adjustment.

In a special embodiment of the invention, the therapeutic device comprises a wound pressure sensor provided under the cover to measure the prevailing distal pressure on the distal end of the drainage tube and a suction pump pressure sensor to measure the suction pump-side pressure p2. The advantage of this is that three pressure measurements are present at three different locations on the device. These measurements make possible the gathering of a large quantity of vacuum therapy operating data. By evaluating the absolute values of the three pressure measurements and the differences between these measured values important input quantities can be obtained for optimized setting and control. Also, all three pressure values can be evaluated together, for example, to calculate an indicator for the trouble-free operation of vacuum therapy.

The inventive task is also solved by a procedure to operate a therapeutic device to promote the healing of a wound, comprising:

an essentially airtight cover around the wound, whereby a fluid-permeable pad is preferably provided between the wood and the cover,

a drainage line connecting the cover with a suction pump can be suctioned on the wound to removed fluids from there, whereby a distal end of the drainage line is indirectly connected with the suction pump via a reservoir to collect the fluid suctioned from the wound,

pressure sensor means for the direct or indirect definition of a prevailing wound pressure in the area between the wound the cover and

a control unit to control wound pressure.

The following procedure is followed to determine wound pressure in the inventive procedure

in a first step, the suction pump is temporarily turned off,

in a second step, a distal pressure is measured with a pressure sensor provided on the distal end of the drainage tube.

Using this procedure it is surprisingly simple to measure the pressure at the wound site, that is, the wound pressure without having to apply a measuring head directly to the wound site or to the pad. In addition, no addition tube is required as probe tube to measure the wound pressure. Instead, according to the invention, the circumstance is used that a uniform pressure is set inside the drainage tube, on the wound-side end of the drainage tube and on the distal end of the drainage tube if the suction pump is turned off and so that no fluidic pressure gradients are created. It cannot be excluded however that leakage in the system or leaks in particular at the wound site can cause slight residual flow. In practice, however, this slight residual flow results in a negligible dynamic pressure gradient in the absence of an extreme leak, which would endanger the success of the therapy in any case.

Therefore after switching off the suction pump, the same pressure will be measured on the distal end of the drainage tube as at the wound site.

After the pressure on the distal end has been measured in this way which equals the wound pressure when applying the inventive procedure, the suction pump can be turned on again if it is immediately necessary to maintain the target pressure. The measurement can, for example, be taken at regular time intervals in order to control the wound pressure.

The procedure described above is further improved in a special embodiment of the invention if a relaxation period is observed between the first and second step, during which a largely uniform pressure is set between the distal and proximal end of the drainage tube. During the relaxation period, the fluidic pressure gradients balance each other out. The relaxation period can be determined in advance by tests.

The inventive task can be solved according to the invention by a procedure to operate a class-appropriate therapeutic device in which to determine the wound pressure at a given suction power of the suction pump a distal pressure is first measured at the pressure sensor provided on the distal end of the drainage tube, and a pressure value is then calculated from a calibration table stored in the control unit using a pair of values taken from the distal pressure and the suction power; this pressure value essentially corresponds to the wound pressure.

An advantage of this procedure to determine wound pressure according to the invention is that it enables continuous measurement of pressure. This is made possible by the fact that a drop in pressure which may be caused by a flow resistance in the drainage tube is considered in the measurement due to the calibration. In this fashion, the wound pressure can be measured advantageously without a pressure sensor being provided at the site of the wound, that is, for example, on the pad. In addition, a probe tube or similar does not have to be run to the wound to enable measurement of the wound pressure. The evaluation of the distal pressure measured by the pressure sensor provided on the distal end of the drainage tube can readily be done. That is where the calibration table is stored which is used to convert the distal pressure into a wound pressure. Using the calibration table, each pair of wound pressure values from the suction power of the suction pump and inside the airtight cover is assigned a distal pressure. On the other hand, the simple assignment of a wound pressure simply to distal pressure without taking into account the pump output in the case of a running pump would not be sufficient due to the suction dependency of the dynamic pressure gradients. The control unit should be provided with suitable interpolation algorithms to calculate the table interim values.

In a special embodiment of the above inventive procedure it is provided that the definition of the calibration table comprises the following steps:

the suction pump is operated with various predetermined constant suctions;

of each suction power the wound pressure is simultaneously measured using a wound pressure sensor provided inside the airtight cover and the distal pressure measured by a distal pressure sensor provided on the distal end of the drainage tube;

the measured wound pressure and distal pressure as well as the associated suction power are entered in the calibration table as a triple value.

This procedure to determine the calibration table is particularly well suited to systematically record the necessary value area.

In addition, the inventive task is similarly solved by the invention with the help of a procedure to operate a class-appropriate therapeutic device which comprises the following steps to report errors in the drainage tube:

simultaneous direct measurement of wound pressure with a running suction pump inside the airtight cover and the distal pressure on the distal end of the drainage tube;

calculation of a difference between the wound pressure and the distal pressure;

comparing the difference with a reference difference;

determining an error in the drainage tube in the event of a deviation from the reference difference by a predetermined threshold value.

This procedure can be used advantageously, for example, to report blockages in the drainage tube. It is also possible with the inventive procedure to determine that the drainage tube is crimped and acts to constrict the pressure. In particular, it is advantageous in this procedure that the wound pressure is measured simultaneously by two different procedures. The direct measurement of wound pressure inside the airtight cover is not influenced by the pressure gradients in the drainage tube influenced by dynamic effects. Conversely, on the other side, the distal pressure on the distal end of the drainage tube is influenced by the dynamic effects in the drainage tube. Measuring both pressure quantities at the same time therefore makes it possible according to the invention to isolate changes in dynamic pressure drops from other effects. That is, if the distal pressure changes without a change in the wound pressure or the suction power of the suction pump, the cause is an error in the drainage tube, such as blockage with wound exudation or similar or a crimping of the tube. This improvement in the metrology of the vacuum therapy device is an enormous advantage in the optimum patient care.

According to an especially advantageous embodiment of the above inventive procedure, the wound pressure is evaluated only on a qualitative basis. To do this, an especially inexpensive sensor—such as a strain gage—can be used to measure wound pressure which is only used as a pressure switch, for example, to detect the exceeding of a minimum negative pressure requires for the therapy. In particular, in the event that the wound bandage unit is designed with the wound pressure sensor as a disposable item, the result is substantial cost savings potential.

### BRIEF DESCRIPTION OF THE DRAWINGS

The invention is described in a preferred embodiment using a drawing, whereby other advantageous details can be found in the drawing figures.

Parts having the same function are labeled with the same reference numbers.

The figures of the drawings show:

FIG. 1 is a schematic representation of the inventive wound healing device and its operating mode.

FIG. 2 is a detailed representation of the inventive wound healing device with the suction tube

### DETAILED DESCRIPTION OF THE PREFERRED EMBODIMENTS

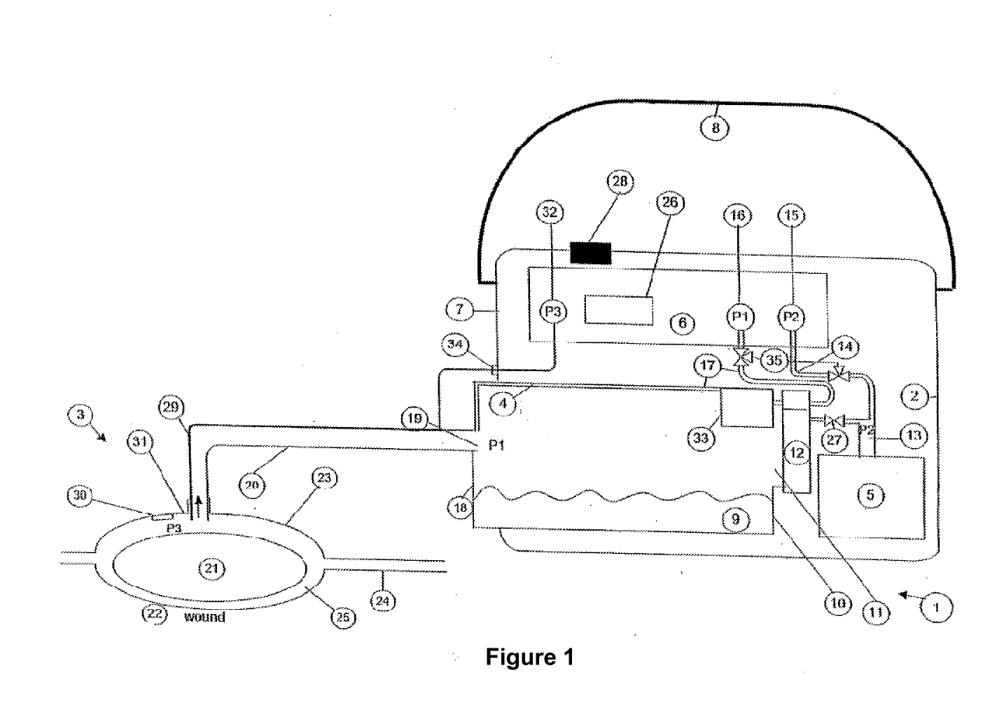

FIG. 1 schematically shows an embodiment of an inventive wound healing device 1. The wound healing device 1 comprises a base unit 2 and a wound dressing unit 3. The base unit 2 contains a replaceable collecting container 4, a suction pump 5 and a control device 6. The base unit 2 is enclosed by a housing 7. A carrying strap 8 is attached to the housing 7.

The collecting container 4 contains a mixture 9 consisting of wound exudation and gelling agent. The collecting container 4 comprises a suction opening 11 on one of the inner walls 10 facing the housing 7. The suction opening 11 is connected to the suction pump 5 by the filter 12 and a suction tube 13. A pump-side probe tube 14 branches off from the suction tube 13. The pump-side probe tube 14 carries the prevailing pressure p2 contained in the suction tube 13 to a suction-pump-side pressure sensor 15 which is located in the control device 6. The control device 6 also contains the distal pressure sensor 16, which interconnects the prevailing pressure p1 prevailing on the distal end of the suction tube 17 via the probe tube 17 and the distal probe tube 36. A 3/2-way valve 35 is installed in both the probe tube 17 and in the probe tube 14. The distal probe tube 36 runs along the replaceable collecting container 4 inside the housing 7 to a suction opening 19 located in the outer wall 18 of the collecting container 4 in the housing 7 of the base unit 2. Also, the distal probe tube 36 can optionally be run through a black box 33.

The wound dressing unit 3 comprises the wound exudation suction tube 20, a sponge 21 and a film foil 23 through which the wound exudation suction tube 20 and the sponge 21 are fastened to skin 24 surrounding the wound 22 in such a way that an airtight connection is created. The sponge 21 is able to absorb and store fluids. Using the stretched film foil 23, the sponge 21 is applied with a certain contact pressure to the wound 22.

Further, a strain gage 30 is fastened in the wound dressing 3 between the film foil 23 and the sponge 21 near the tube connector 31. The strain gage 30 is used to measure the wound pressure on the wound 22. The strain gage 30 with connected to an electrical cable 29. The electrical cable 29 is connected to the outer edge of the suction tube 20 and in this way connected to the evaluation device 32 for the wound pressure P3 by means of a plug-type connector 34 in the housing 7 of the base unit 2. The evaluation device 32 is part of the control device 6.

The collecting container 4, the wound exudation suction tube 20, the sponge 21 and the film foil 23 are respectively sterile and replaceable for disposable use. The wound exudation is located in the wound 22.

To use the inventive wound healing device 1, the sponge 21 is introduced into the wound 22. One end of the wound exudation suction tube 20 is connected to the sponge 21, and then both the wound exudation suction tube 20 and the sponge 21 are affixed to the wound 22 with the help of the film foil 23 in such a way that the system is made airtight.

The other end of the wound exudation suction tube 20 is also connected to the suction opening 19 contained in the outer wall 18 of the collecting container 4 in an airtight fashion. Due to the property of the sponge 21 to absorb and store fluids, the wound exudation 25 is removed from the wound 22 into the sponge 21. The wound exudation quantity that exceeds the storage capacity of the sponge 21 is suctioned into the collecting container 4 through the wound exudation suction tube 20. The negative pressure needed for the suction is produced by the suction pump 5.

For this purpose the suction tube 13 of the suction pump 5 interconnects the collecting container 4. The filter 12 arranged between the collecting container 4 prevents the wound exudation 25 from entering the suction tube 13, the suction pump 5, the suction-pump-side probe tube 14 or even the suction-pump-side pressure sensor 15.

The excess wound exudation 25 collected in the collecting container 4 in the manner described above is thickened by a gelling agent. This effectively prevents the moistening of the filter 12 even when the base unit 2 is moved. As a result, for example, the filter 12 is prevented from being moistened and germs are prevented from getting into the control device 6. Also, it is possible to dispose of the potentially contaminating wound fluid in the solid aggregate state in a more hygienic fashion.

The negative pressure produced by the suction pump 5 is continuously received on the one hand by the suction-pump-side pressure sensor 15 and by the wound-side strain gage 30 on the other and also via the distal pressure sensor 16, which measures the distal pressure p1. Then, the pressure p3 taken from the wound-side strain gage 30, the pressure p2 taken from the suction-pump-side pressure sensor 15 and the pressure p1 taken from the distal end of the suction tube 20 are used in the microprocessor of the control device 6 as input data for control.

In order to maintain a moist wound environment, the pressure level in the wound dressing unit 3 is set according to the individual needs of the patient, the properties of the wound 22 and according to the criteria of compatibility (pain by the patient). The setting is done using a software menu mode on a display unit 26 on the microprocessor-controlled control device 6. The software menu mode makes it possible for the user to select different languages. Also, the desired control program for the negative pressure on wound dressing unit 3 can be selected using the software menu mode. The microprocessor of the control device 6 processes the signal of the wound-side strain gage 30, that is pressure signal p3, of suction-pump-side pressure sensor 15, that is pressure signal p2 and distal pressure sensor 16, that is the distal pressure p1—according to the selected control algorithm at output readings. The output readings are fed by the control device 6 to the control valve 27 and, optionally, also directly to the suction pump 5.

Assisted by the control valve 27, which is located in the suction tube 13 between the suction pump 5 and the filter 12, various pressure control programs can be physically realized as the control valve 27 can be opened and closed quickly according to the control signals of the control device 6.

The user can also run a fixed pressure control program through the display unit and the software menu mode. Assessing the pressure signals p1, p2 and p3 sent from the pressure sensors 15, 16, 30/32, the control device 6 influences the valve 27 and/or the suction pump 5 as outputs for the pressure areas and control times. In term of control, only slightly deviations are permitted of the actual value from the target value.

With the carrying strap 8, the base unit 2 can be carried by the patient on whose wound 22 the wound dressing unit 3 is applied. This makes the patient mobile even during use of the inventive wound healing device 1.

For service and for patient documentation purposes, the control device 6 is equipped with a computer interface 28. It can be used, for example, to input new control algorithms using an external computer or to forward patient information to a central administration.

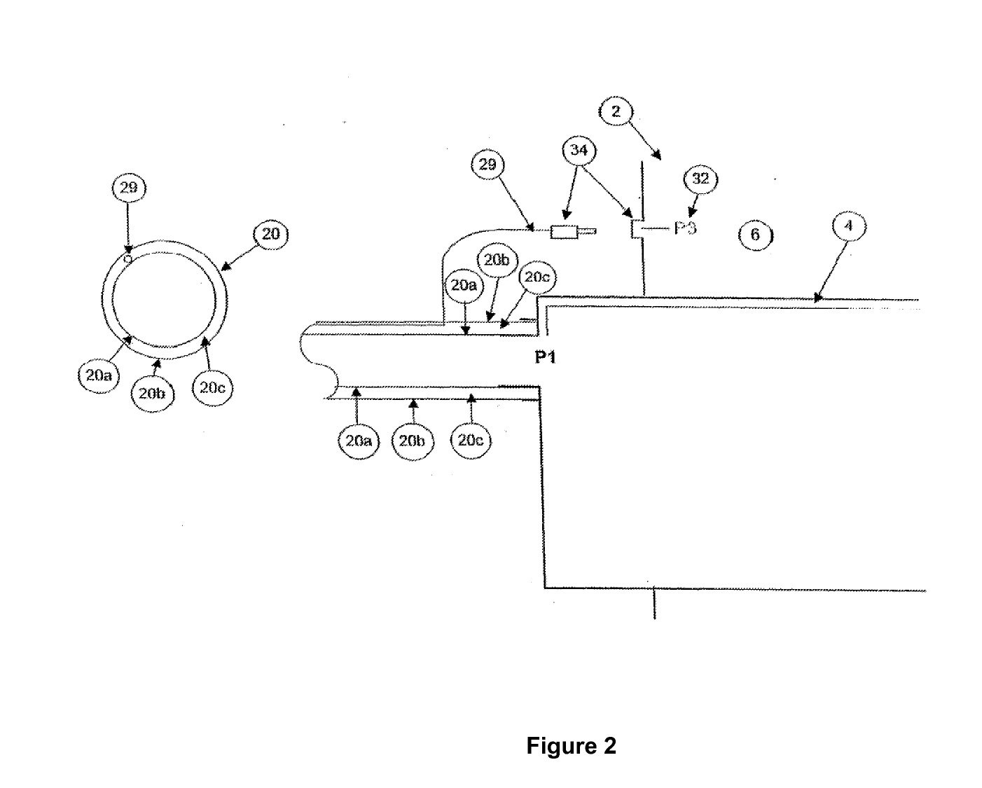

FIG. 2 depicts a preferred option of connecting the electrical cable 29 to the base unit 2. As shown in the figure, the suction tube 20 represented in cross section comprises an interior 20a and an exterior 20b tube wall. The electrical cable 29, which connects the strain gage 30 with the control unit 6 in the base unit 2 is run in the cavity 20c between the interior 20a and an exterior 20b tube wall. The electrical cable 29 runs out of the cavity 20c near the base unit 2 and it is connected with the control device in the base unit 2 via the plug-type connector 34. In this fashion, the signal of the strain gage 30 is sent to the evaluation device 32 for the wound pressure p3.

The device can use the following different procedures to measure wound pressure:

Firstly, the pressure can be taken directly using the strain gage 30 on the wound site around the airtight covering of the wound. The evaluation of the measurement p3 for the wound pressure of the strain gage 30 is done using the evaluation device 32 inside the control device 6.

Further, the wound pressure can be determined by measuring the distal pressure p1 with the distal pressure sensor 16, which reads the pressure on the distal end of the suction tube 20. This method takes into account the dynamic pressure gradient caused by the suction flow by calibrating with the help of the control device 6. The calibration is done by evaluating the wound pressure p3 measured by the strain gage 30 in conjunction with the equivalent values for p1. This pair of values must be produced by different pump powers and input into the calibration curve. After the calibration curve is determined, the strain gage 30 on the wound site is no longer required. Therefore, normally, the wound dressing could be supplied without measuring equipment. Only for calibration purposes could a calibration wound dressing be used which comprises a strain gage or other pressure sensors. In this way the manufacturing costs of the normally used disposable wound dressings can be kept advantageously low.

Finally, the wound pressure can also be determined by measuring the distal pressure p1 without having to take into account dynamic effects. In this case, before each measurement the suction pump 5 can be switched off and a relaxation period can be observed. During the relaxation period, a stationary condition is created in which the pressure p3 at the wound site which is the same as the pressure p1 at the distal end of the drainage tube 20. In this case, a simple measurement of the pressure p1 by the pressure sensor 16 via the probe tube 36 already provides the sought wound pressure. To undertake control, this reading should be taken at regular intervals.

In addition, the described processes for measuring wound pressure can be used simultaneously. On the one hand, this can result in an improvement in the reliability of the measurement. Also, error diagnosis procedures can also be conducted in this fashion to, for example, determine a blockage of the drainage tube. Blockage exists when there is a change of the pressure p1 with a constant measurement p3, whereby p1 can be measured either with the dynamic or the static methods described above.

In order to determine a blockage of the filter 12, the prevailing pressure p2 in the suction tube 13 can be measured on the suction side of the suction pump 5 with the help of the suction-pump-side pressure sensor 15 and at the same time with the distal pressure sensor 16 via the probe tube 17 and the prevailing pressure p1 on the end of the suction tube 13 distal probe tube 36. The difference between p1 and p2 assessed in the control device 6 is then correlated with the degree of blockage of the filter. When a certain degree of blockage is reached, the user can be requested to change the filter.

Further, the measurement of the pressure p2 can also be used to measure the suction power of the suction pump 5.

Therefore a device is proposed to promote wound healing entailing a significant improvement in metrology and which results in a major improvement over conventional devices and which makes possible more reliable control.

### LIST OF DRAWING REFERENCES

1 Wound healing device
2 Base unit
3 Wound dressing unit
4 Collecting container
5 Suction pump
6 Control device
7 Housing
8 Carrying strap
9 Mixture
10 Inside wall
11 Suction opening
12 Filter
13 Suction tube
14 Suction-pump-side probe tube
15 Suction-pump-side pressure sensor
16 Wound-side pressure sensor
17 Wound-side probe tube
18 Outer wall
19 Suction opening
20 Wound exudation suction tube
20a Interior tube wall
20b Exterior tube wall
20c Cavity
21 Sponge
22 Wound
23 Film foil
24 Skin
25 Wound exudation
26 Display unit
27 Control valve
28 Computer interface
29 Electrical cable
30 Strain gage
   31 Tube connector
   32 P3 evaluation device
   33 Black box
   34 Plug-type connector
   35 3/2-way valve
   36 Distal probe tube

1. A therapeutic device to promote the healing of a wound, comprising:
a. cover that is positioned around the wound, and a liquid-permeable pad provided between the wound and the cover;
b. a drainage line coupling the cover with a suction pump that applies a suction to the wound to draw fluids therefrom, wherein a distal end of the drainage line is coupled to the suction pump through a reservoir to collect the fluid suctioned from the wound;
c. a pressure sensor that identifies a wound pressure in the area between the wound and the cover; and
d. a control unit that regulates the wound pressure;
and wherein the pressure sensor comprises at least one sensor to measure a prevailing distal pressure on a distal end of the drainage tube.

2. A therapeutic device to promote the healing of a wound, comprising:
a. cover that is positioned around the wound, and a liquid-permeable pad provided between the wound and the cover;
b. a drainage line coupling the cover with a suction pump that applies a suction to the wound to draw fluids therefrom, wherein a distal end of the drainage line is coupled to the suction pump through a reservoir to collect the fluid suctioned from the wound;
c. a pressure sensor that identifies a wound pressure in an area between the wound and the cover and a control unit to regulate the wound pressure;
wherein the pressure sensor comprises at least one wound pressure sensor provided under the cover and a connection that transfers measurements to the control unit.

3. The therapeutic device of claim 2, wherein the at least one wound pressure sensor is designed as a strain gage.

4. The therapeutic device of claim 3, wherein the strain gage (30) is provided in an operating connection with a connector (31) to connect the cover (21) to the drainage line (20).

5. The therapeutic device of claim 2, wherein a control pump is provided in addition to the suction pump.

6. The therapeutic device of claim 2, wherein the pressure sensor measures a prevailing distal pressure on a distal end of a draining tube and a suction pump pressure sensor records a suction-pump-side pressure.

7. A method for operating a therapeutic device to promote the healing of a wound comprising:
a. positioning cover around the wound, wherein a liquid-permeable pad is provided between the wound and cover;
b. draining fluids from the wound using a drainage line coupled to the cover with a suction pump, wherein a distal end of the drainage line is coupled to the suction pump through a reservoir to collect the fluid suctioned from the wound;
c. sensing a wound pressure in the area between the wound and the cover and;
d. regulating a wound pressure; and
wherein the wound pressures is sensed comprising the following steps:
temporarily switching the suction pump off;
measuring a distal pressure with a pressure sensor provided on the distal end of the drainage tube.

8. The method of claim 7, wherein a relaxation period is observed between switching the pump off and measuring the distal pressure and during which a largely uniform pressure is set between the distal and the proximal end of the drainage tube.

9. A method for operating a therapeutic device to promote the healing of a wound comprising:
a. positioning cover around the wound, wherein a liquid-permeable pad is provided between the wound and cover;
b. draining fluids from the wound using a drainage line coupled to the cover with a suction pump, wherein a distal end of the drainage line is coupled to the suction pump through a reservoir to collect the fluid suctioned from the wound;
c. sensing a wound pressure in the area between the wound and the cover and;
d. regulating a wound pressure; and
wherein the wound pressure at a given suction power of the suction pump is determined by measuring a distal pressure on the distal end of the drainage tube and by calculating a pressure value from a pair of values from a calibration table stored in a control unit and the given suction power being essentially equal to the wound pressure.

10. The method of claim 9, wherein defining the calibration table comprises the steps of:
a. operating the suction pump at various predetermined constant suction powers;
b. measuring a wound pressure for each of the predetermined constant suction powers using a wound pressure sensor provided inside the cover and the distal pressure using a distal pressure sensor provided on the distal end of the drainage tube,
c. entering the measured wound pressure and distal pressure as well as the relevant suction power as a three-part value in the calibration table.

11. A method for operating a therapeutic device to promote the healing of a wound comprising:
a. positioning cover around the wound, wherein a liquid-permeable pad is provided between the wound and cover;
b. draining fluids from the wound using a drainage line coupled to the cover with a suction pump, wherein a distal end of the drainage line is coupled to the suction pump through a reservoir to collect the fluid suctioned from the wound;
c. sensing a wound pressure in the area between the wound and the cover and;
d. regulating a wound pressure; and
wherein an error in the drainage tube is found using a method comprising the steps of:
e. measuring the wound pressure with a running suction pump inside the cover and the distal pressure on the distal end of the drainage tube;
f. calculating a difference between the wound pressure and the distal pressure;
g. comparing the difference with a reference difference;
h. finding an error in the drainage tube using a deviation of the reference difference by a predetermined threshold value.

12. The method of claim 12 wherein the wound pressure is evaluated only qualitatively.
